(19) United States
(12) Patent Application Publication
(10) Pub. No.: US 2018/0187029 A1
(43) Pub. Date: Jul. 5, 2018

(54) COMPOSITION FOR FORMING HIGH RELEASE AND LOW FRICTION FUNCTIONAL COATINGS

(71) Applicant: WHITFORD CORPORATION, Elverson, PA (US)

(72) Inventors: Matteo Sperindio, Brescia (IT); Paolo Luvié, Codogno (IT); Leonard W. Harvey, Downington, PA (US)

(73) Assignee: WHITFORD CORPORATION, Elverson, PA (US)

(21) Appl. No.: 15/739,374

(22) PCT Filed: Jul. 7, 2016
(86) PCT No.: PCT/US16/41228

§ 371 Date: Dec. 22, 2017

Related U.S. Application Data
(60) Provisional application No. 62/190,984, filed on Jul. 10, 2015.

(51) Int. Cl. C09D 7/20; C09D 7/61; C09D 7/65; C09D 181/04; C09D 181/06; C09D 179/08; C07C 235/06; B05D 5/08; A47J 36/02

(52) U.S. Cl. CPC C09D 7/20; C09D 7/61; C09D 7/65; C09D 181/04; C08K 3/04; C09D 179/08; C07C 235/06; B05D 5/083; A47J 36/02; C09D 181/06

(57) ABSTRACT

The present disclosure provides a composition for forming a coating including at least one dry lubricant material, at least one binder resin, and at least one β-alkoxypropionamide solvent. The composition can be applied to a wide variety of rigid and flexible substrates. A process for making articles is also provided.

### CROSS-REFERENCE TO RELATED APPLICATIONS

This application claims the benefit under Title 35, U.S.C. § 119(e) of U.S. Provisional Patent Application No. 62/190,984, entitled “Composition for Forming High Release and Low Friction Functional Coatings”, filed on Jul. 10, 2015, the entire disclosure of which is expressly incorporated by reference herein.

### FIELD

The present disclosure relates to compositions for forming coatings, and more particularly, to compositions that include a β-alkoxypropionamide-based solvent system and one or more functional components.

### BACKGROUND

Functional coatings that provide non-stick or reduced friction properties are often prepared from one or more dry lubricants, such as fluoropolymers, graphite, molybdenum dislulphide, boron nitride, silicones, and the like, embedded in or coupled to a binder medium. Typical binders include engineering polymers, such as polyamideimide, polyarylsulfone, polyphenylensulfide, polyetherimide, polyimide, polyetherketones, and the like.

Such engineering polymers typically include aromatic structural fragments and the presence of heteroatoms that are responsible for the high performances in terms of temperature, mechanical and chemical resistance. However, these structures also result in limited solubility of the polymers in conventional solvents. Typically, a narrow class of special solvents among the polar aprotic group is used in order to be able to work with these polymers in the coating preparation.

When attempting to prepare an organic coating that provides reduced friction or non-stick properties there are several available technologies, including water borne and solvent borne coatings. These coatings may be used in particularly harsh environmental conditions that require contact with solvents, resistance to wear, and resistance to high temperatures. Typical coatings include one or more dry lubricants or non-stick agents, such as fluoropolymers, graphite, molybdenum or tungsten disulfide, hexagonal boron nitride, polydimethyl siloxanes and the like. The dry lubricant or non-stick agent is typically coupled to one or more organic polymers having high thermal, mechanical and chemical resistance for the service or the manufacturing of the coating. These organic polymers may be referred to as engineering polymers, and include a family of specialty polymers often prepared from monomers including aromatic rings and heteroatoms such as nitrogen or sulfur. Some of these polymers are thermosetting, others are thermoplastic. Non-limiting examples of engineering polymers include polyamide-imide resin, polyimide resin, polyether imide resins, polyether sulfone resins, polyether ether ketone resins, polyphenylene sulfide resins, and the like.

Most engineering polymers are insoluble in traditional organic solvents and require particular solvents, such as polar aprotic solvents. Exemplary solvents include gamma-butyrolactone, N-methyl-2-pyrrolidone, N-ethyl-2-pyrrolidone, N,N-dimethylformamide, furfuryl alcohol, and the like. Traditionally, for the formulation of reduced friction or non-stick coatings, the most widely used solvent is N-methyl, 2-pyrrolidone. However, some traditional polar aprotic solvents are the subject of increased labeling and environmental regulations due to possible toxicity or psychotropic effects of these solvents.

Other solvents, such as dimethylsulfoxide (DMSO), are capable of partially dissolving some engineering polymers, but provide other technical limitations such as high melting point, hygroscopic effect, strong smell, and possible safety concerns such as being a strong allergenic, and behaving as vehicles for substances through skin adsorption.

Improvements in the foregoing are desired.

### SUMMARY

The present disclosure provides compositions for forming coatings, such as functional non-stick or reduced friction coatings. In one embodiment, the composition includes at least one β-alkoxypropionamide solvent, together with at least one dry lubricant material and at least one binder resin.

In one exemplary embodiment, a composition for forming a coating is provided, which includes at least one functional additive; at least one binder; and at least one 3-alkoxypropionamide solvent of the formula:

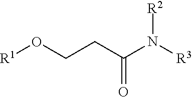

wherein R¹ is a C₁ to C₈ alkyl, and

R² and R³ are independently selected from hydrogen, C₁ to C₆ alkyl, C₁ to C₆ alkoxy, C₁ to C₆ alkoxyalkyl, or glycidyl. Alternatively, R¹ is a C₁ to C₄ alkyl, and R² and R³ are independently selected from H or C₁ to C₄ alkyl.

In a more particular embodiment, the β-alkoxypropionamide solvent is selected from the group consisting of 3-methoxy-N,N-dimethylpropanamide and 3-buthoxy-N,N-dimethylpropanamide. In a further embodiment, the at least one functional additive includes at least one additive selected from the group consisting of: graphite, molybdenum disulphide, hexagonal boron nitride, fluoropolymers, and silicone-based materials. In a still further embodiment, the at least one functional additive includes at least one fluoropolymer selected from the group consisting of: polytetrafluorethylene (PTFE); fluorinated ethylene-propylene (FEP); perfluoroalkoxy polymer (PFA); perfluoro methylalkoxy polymer (MFA); polyvinylidene fluoride (PVDF); polyethylenetetrafluoroethlene (ETFE); polyethylenechlorotrifluoroethylene (ECTFE); and polymers of tetrafluoroethylene, hexafluoropropylene and vinylidene fluoride (THV). In a still further embodiment, the at least one functional additive includes polytetrafluorethylene (PTFE).

In a further embodiment, the at least one binder includes at least one engineering polymer selected from the group consisting of: polyphenylene sulfide (PPS); polyetheretherketone (PEEK); polysulfone (PESU); polyimide (PI); polyamide-imides (PAI); and polyetherimide (PEI). In a further embodiment, the composition further includes at least one additional component selected from the group consisting of pigments and colorants; non-β-alkoxypropionamide solvents; functional fillers; defoamers; surface wetting agents; flow agents; pigment wetting additives; thickeners; fillers; additives that change the electric conductivity of the composition; pH correctors; and flash rust inhibitors.

In another exemplary embodiment, a method for forming a coating is provided. The method includes the steps of: providing a composition, wherein the composition comprises at least one functional additive, at least one binder, and at least one β-alkoxypropionamide solvent of the formula:

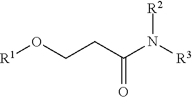

wherein R¹ is a C₁ to C₈ alkyl, and R² and R³ are independently selected from hydrogen, C₁ to C₆ alkyl, C₁ to C₆ alkoxy, C₁ to C₆ alkoxyalkyl, or glycidyl; applying the composition to a substrate; and curing the composition to produce a coating. Alternatively, R¹ is a C₁ to C₄ alkyl, and R² and R³ are independently selected from H or C₁ to C₄ alkyl.

In a more particular embodiment, the β-alkoxypropionamide solvent is selected from the group consisting of 3-methoxy-N,N-dimethylpropanamide and 3-buthoxy-N,N-dimethylpropanamide.

In a further embodiment, the curing step may include heating the coating at a temperature of 400° C. to 450° C. for a time from 3 minutes to 20 minutes, and/or may include drying in air at ambient temperature. In a still further embodiment, the method may further include the additional steps of: heating the applied composition and substrate to form a first dried layer; and applying a second composition to the first dried layer, wherein the second composition comprises at least one solvent, at least one binder, and at least one functional additive; wherein said curing step comprises curing the first dried layer and the second composition to produce a coating.

In a further exemplary embodiment, a coated article including a substrate coated with a coating is provided. The coating is formed from a composition including at least one functional additive; at least one binder; and at least one β-alkoxypropionamide solvent of the formula:

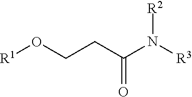

wherein R¹ is a C₁ to C₈ alkyl, and

R² and R³ are independently selected from hydrogen, C₁ to C₆ alkyl, C₁ to C₆ alkoxy, C₁ to C₆ alkoxyalkyl, or glycidyl. Alternatively, R¹ is a C₁ to C₄ alkyl, and R² and R³ are independently selected from H or C₁ to C₄ alkyl.

In another embodiment, the substrate is selected from the group consisting of cookware, bakeware, molds, small electrical appliances, fasteners, reprographic rollers, glasscloth, architectural fabrics, as well as heat sealing belts, circuit boards, cooking sheets, tenting fabrics, staple fiber, fiberfill, yarn, thread, textiles, nonwoven fabric, wire cloth, ropes, belting, cordage, and webbing. In a particular embodiment, the coated article is an article of cookware, and in a still further embodiment, the coating has a dry film thickness from 5 microns to 30 microns.

In a more particular embodiment, the β-alkoxypropionamide solvent is selected from the group consisting of 3-methoxy-N,N-dimethylpropanamide and 3-buthoxy-N,N-dimethylpropanamide.

The above mentioned and other features of the invention, and the manner of attaining them, will become more apparent and the invention itself will be better understood by reference to the following description of embodiments of the invention taken in conjunction with the accompanying drawings.

### BRIEF DESCRIPTION OF THE FIGURES

The disclosure is explained in greater detail below in reference to the figures. In the figures:

FIG. 1 is related to Example 1 and shows a comparison of the viscosity of an example prepared with a β-alkoxypropionamide solvent, and a comparative example prepared with an N-ethyl-2-pyrrolidone solvent.

FIG. 2A is related to Example 1 and shows a surface tension test for a comparative example prepared with an N-ethyl-2-pyrrolidone solvent.

FIG. 2B is related to Example 1 and shows a surface tension test for an example prepared with an β-alkoxypropionamide solvent.

FIG. 3 is related to Example 1 and shows surface tension versus drop area for an example prepared with a β-alkoxypropionamide solvent, and a comparative example prepared with an N-ethyl-2-pyrrolidone solvent.

FIG. 4 is related to Example 2 and shows a comparison of particle size distribution for an example prepared with a β-alkoxypropionamide solvent, and a comparative example prepared with an N-ethyl-2-pyrrolidone solvent.

FIGS. 5A-5C are related to Example 2 and show a surface ATR-FTIR map of a comparative example prepared with an N-ethyl-2-pyrrolidone solvent showing PTFE agglomeration and strong PESU saturation.

FIGS. 6A-6C are related to Example 2 and show a surface ATR-FTIR map of an example prepared with an β-alkoxypropionamide solvent showing PTFE even distribution and low PESU saturation, replaced by stronger signals of PTFE.

FIG. 7 is related to Example 3, and shows shear stability results for a two component blend of a PTFE dispersion and a solvent.

FIG. 8 is related to Example 3 and shows shear stability results for a three component blend of a PTFE dispersion, a PAI, and a solvent.

FIG. 9 is related to Example 4 and shows shear stability results for a three component blend of a PTFE dispersion, a PAI, and a solvent.

### DETAILED DESCRIPTION

The present disclosure provides compositions for forming functional coatings.

In some embodiments, the compositions include one or more β-alkoxypropionamide solvents. Compared to typical solvents, β-alkoxypropionamide solvents show no or a reduced toxicity, low flammability, corrosion protection, and favorable features for the formulation of compositions for reduced friction and non-stick coatings.

In an exemplary embodiment, a composition for forming a functional coating comprises at least one functional additive, at least one binder, and at least one β-alkoxypropionamide solvent.

In some embodiments, the composition may optionally include one or more additional components. Exemplary additional components include pigments and colorants, additional solvents, functional fillers, additives, and acidic or alkaline additives.

### 1. Functional Additives

The composition includes one or more functional additives, such as dry lubricants or solid lubricants. Dry lubricants are materials that, despite being in the solid phase, are able to reduce friction between two surfaces sliding against each other without the need for a liquid medium. Dry lubricants offer lubrication at temperatures higher than those at which liquid and oil-based lubricants operate. Dry lubricants may be used in applications such as locks or dry lubricated bearings. Such materials can operate up to 350° C. (662° F.) in oxidizing environments and even higher in reducing/non-oxidizing environments (molybdenum disulfide up to 1100° C., 2012° F.). Without wishing to be held to any theory, it is believed that the low-friction characteristics of most dry lubricants are due to a layered structure on the molecular level with weak bonding between layers. Such layers are able to slide relative to each other with minimal applied force, thus giving them their low friction properties. Other dry lubricants, such as polytetrafluroethylene, have non-lamellar structures that function well as dry lubricants in some applications. Exemplary dry lubricants include, but are not limited to, graphite, molybdenum disulphide, hexagonal boron nitride, fluoropolymers, and silicone-based materials.

In one exemplary embodiment, the composition comprises, on a wet basis, one or more functional additives in an amount from as little as 0.5 wt. %, 1 wt. %, 5 wt. %, as great as 25 wt. %, 50 wt. %, 70 wt. %, or within any range defined between any two of the foregoing values.

In one exemplary embodiment, the coating formed from the composition comprises, on a dry basis, one or more functional additives in an amount from as little as 3 wt. %, 5 wt. %, 10 wt. %, as great as 50 wt. %, 75 wt. %, 95 wt. %, or within any range defined between any two of the foregoing values.

a. Graphite

In one exemplary embodiment, the functional additive comprises graphite. Graphite has been used for lubrication in air compressors, food industry, railway track joints, open gear, ball bearings, engine piston skirts, machine-shop works, and similar applications. Graphite is also used for lubricating locks, as liquid lubricants allow particles to get stuck in the lock, thereby worsening the problem. Graphite is structurally composed of planes of polycyclic carbon atoms that are hexagonal in orientation. The distance of carbon atoms between planes is longer and therefore the bonding is weaker. Graphite is suitable for lubrication in air. At least some water vapor is typically necessary for graphite lubrication. The adsorption of water by graphite reduces the bonding energy between the hexagonal planes of the graphite to a lower level than the adhesion energy between a substrate and the graphite. Because water vapor is a requirement for lubrication, graphite is typically not effective in vacuum. In an oxidative atmosphere, graphite is effective at high temperatures up to 450° C. continuously and can withstand much higher temperature peaks. Graphite is characterized by two main types: natural and synthetic. Synthetic graphite is a high temperature sintered product and is characterized by its high purity of carbon (99.5-99.9 wt. %). The primary grade synthetic graphite can approach the good lubricity of quality natural graphite. Natural graphite is derived from mining. The quality of natural graphite varies as a result of the ore quality and post mining processing of the ore. The end product is graphite with a content of carbon (high grade graphite 96-98 wt. % carbon), sulfur, SiO₂ and ash. The higher the carbon content and the degree of graphitization (high crystalline) the better the lubricity and resistance to oxidation. For applications where only a minor lubricity is needed and a more thermally insulating coating is required, amorphous graphite (80% carbon) is the most useful.

b. Molybdenum Disulfide

In one exemplary embodiment, the functional additive comprises molybdenum disulphide. Molybdenum disulphide (MoS₂) has been used in CV joints and space vehicles. Molybdenum disulphide is mined from some sulfide-rich deposits and refined in order to achieve a purity suitable for lubricant applications. Like graphite, MoS₂ has a hexagonal crystal structure with the intrinsic property of easy shear. MoS₂ lubrication performance often exceeds that of graphite and is effective in vacuum as well whereas graphite is typically not. Lubrication of MoS₂ is typically limited to a temperature of 400° C. due to oxidation. The particle size and film thickness of MoS₂ are typically matched to the surface roughness of the substrate. In some situations, large particles of MoS₂ may result in excessive wear by abrasion caused by impurities in the MoS₂, while small particles may result in accelerated oxidation.

c. Hexagonal Boron Nitride

In one exemplary embodiment, the functional additive comprises hexagonal boron nitride. Hexagonal boron nitride has been used for lubrication in space vehicles. Also called “white graphite,” hexagonal boron nitride is a ceramic powder lubricant. It has a high temperature resistance of 1200° C. service temperature in an oxidizing atmosphere. Furthermore, boron has a high thermal conductivity. Boron is available in two chemical structures, i.e. cubic and hexagonal where the hexagonal is typically used for lubrication.

d. Fluoropolymers

In one exemplary embodiment, the functional additive comprises one or more fluoropolymers. Fluoropolymers are widely used as additives in lubricating oils and greases. Fluoropolymers possess the unique feature of being able to film-form by sintering (melt and flow process), and therefore they may provide in some circumstances the features of binders, besides being a functional additive.

Stable unflocculated dispersions of fluoropolymers, such as polytetrafluorethylene (PTFE), in oil or water can be produced. Contrary to the other solid lubricants discussed, typical fluoropolymers, such as PTFE, do not have a layered structure. The macro molecules of PTFE slip easily along each other, similar to lamellar structures. PTFE possess very low coefficients of static and dynamic friction, down to 0.04. Operating temperatures are typically limited to about 260° C.

Besides the reduced friction feature, PTFE provides reduced friction, as well as low surface tension, as low as 17 mN/m. The low surface tension makes PTFE an outstandingly good material for the preparation of non-stick coatings, where the main characteristic is the ability to prevent adhesion to the coating surface of other materials, like food or glues, even at temperatures as high as 260° C.

In one exemplary embodiment, the functional additive comprises one or more fluoropolymers selected from the group consisting of: polytetrafluorethylene (PTFE); fluorinated ethylene-propylene (FEP); perfluoroalkoxy polymer (PFA); perfluoro methylalkoxy polymer (MFA); polyvinylidene fluoride (PVDF); polyethylenetetrafluoroethlene (ETFE); polyethylenechlorotrifluoroethylene (ECTFE); and polymers of tetrafluoroethylene, hexafluoropropylene and vinylidene fluoride (THV).

In a more particular embodiment, the functional additive comprises polytetrafluorethylene (PTFE). In some embodiments, the PTFE may include a small amount of modifying co-monomer, in which case the PTFE is a co-polymer known in the art as “modified PTFE” or “trace modified PTFE”. Examples of the modifying co-monomer include perfluoropropylvinylether (PPVE), other modifiers, such as hexafluoropropylene (HFP), chlorotrifluoroethylene (CTFE), perfluorobutylethylene (PFBE), or other perfluoroalkylvinylethers, such as perfluoromethylvinylether (PMVE) or perfluoroethylvinylether (PEVE). The modifying co-monomer will typically be present in an amount less than 1% by weight, for example, based on the weight of the PTFE.

e. Silicone-Based Materials

In one exemplary embodiment, the functional additive comprises one or more silicone-based materials. Silicone-based materials are sometimes also used as functional additives in the formulation of non-stick and reduced friction coatings mainly with the purpose of providing enhanced surface gliding and release properties. Accordingly to their formulation their appearance can range from tough glassy solid materials to rubbery polymers to viscous oily fluids. They are generally very stable to temperature and chemical attacks, which makes them suitable candidates for high performance applications as additives in harsh environments. Exemplary silicone-based materials include polydimethylsiloxanes, hydroxyl-terminated polydimethylsiloxanes, and the like.

### 2. Binder

The composition includes one or more binders. The term “binder” refers generally to a polymer that has the ability to film-form and therefore to bind into its polymeric film other materials, generally referred to as fillers. For coatings, and in particular low friction or “non-stick” coatings of the type used with cookware, for example, the binder also promotes adhesion of the coating to a metallic substrate. In the current technology of functional coatings that provide reduced friction and non-stick properties there are several binders that can be used accordingly to the performances required to the coating in use. Typical binders include organic polymers, such as engineering polymers. Engineering polymers are a group of plastic materials that typically have better mechanical and/or thermal properties than the more widely used commodity plastics (such as polystyrene, PVC, polypropylene and polyethylene).

In some embodiments, the binder is an engineering polymer that is either thermosetting or thermoplastic, and that has a glass transition temperature (T_{g}) or a melting point of 180° C. or higher. Exemplary engineering polymers include, for example, polyphenylene sulfide (PPS); polyetheretherketone (PEEK); polysulfone (PESU); polyimide (PI); polyamide-imides (PAI); and polyetherimide (PEI).

In one exemplary embodiment, the composition comprises, on a wet basis, one or more binders in an amount from as little as 3 wt. %, 5 wt. %, 10 wt. %, as great as 25 wt. %, 50 wt. %, 70 wt. %, or within any range defined between any two of the foregoing values.

In one exemplary embodiment, the coating formed from the composition comprises, on a dry basis, one or more binders in an amount from as little as 5 wt. %, 10 wt. %, 25 wt. %, as great as 50 wt. %, 75 wt. %, 97 wt. %, or within any range defined between any two of the foregoing values.

a. Polyphenylene Sulfide (PPS)

In one exemplary embodiment, the binder comprises polyphenylene sulphide. Polyphenylene sulfide (PPS) is an organic polymer consisting of aromatic rings linked with sulfides. Polyphenylene sulfide is an engineering plastic, commonly used as a high-performance thermoplastic. PPS can be molded, extruded, or machined to high tolerances. In its pure solid form, it may be opaque white to light tan in color. Maximum service temperature is typically 218° C. (424° F.). PPS is not known to dissolve in any solvent at temperatures below about 200° C. (392° F.).

b. Polyetheretherketone (PEEK)

In one exemplary embodiment, the binder comprises polyetheretherketone (PEEK). Polyetheretherketone (PEEK) is a semicrystalline thermoplastic with excellent mechanical and chemical resistance properties that are typically retained to high temperatures. The processing conditions used to mold PEEK can influence the crystallinity and mechanical properties of the resulting material. The Young's modulus of PEEK is 3.6 GPa and its tensile strength 90 to 100 MPa. PEEK melts around 343° C. (662° F.). Some typical grades of PEEK have a useful operating temperature of up to 250° C. (482° F.). The thermal conductivity of PEEK increases nearly linearly versus temperature between room temperature and solidus temperature. PEEK is highly resistant to thermal degradation as well as attack by both organic and aqueous environments. PEEK is attacked by halogens and strong Brønsted and Lewis acids as well as some halogenated compounds and aliphatic hydrocarbons at high temperatures. PEEK dissolves completely in concentrated sulfuric acid at room temperature.

c. Polysulfone (PESU)

In one exemplary embodiment, the binder comprises polysulfone (PESU). Polysulfone (PESU) describes a family of thermoplastic polymers containing the subunit aryl-SO₂-aryl, the defining feature of which is the sulfone group. These polymers are known for their toughness and stability at high temperatures. Due to the high cost of raw materials and processing, polysulfones are typically used in specialty applications and often are a superior replacement for polycarbonates. These polymers are typically rigid, high-strength, and transparent, retaining these properties between about 100° C. and 150° C. PESU has very high dimensional stability; the size change when exposed to boiling water or 150° C. air or steam is typically below 0.1%. The glass transition temperature T_{g} of PESU is 185° C. Polysulfone is highly resistant to mineral acids, alkali, and electrolytes, in pH ranging from 2 to 13. PESU is typically resistant to oxidizing agents, therefore it can be cleaned by bleaches. PESU is typically also resistant to surfactants and hydrocarbon oils. PESU is typically not resistant to low-polar organic solvents (e.g. ketones and chlorinated hydrocarbons), and aromatic hydrocarbons. Mechanically, polysulfone has high compaction resistance, recommending its use under high pressures. PESU is also stable in aqueous acids and bases and many non-polar solvents; however, it is typically soluble in dichloromethane and N-methyl pyrrolidone.

d. Polyimide (PI)

In one exemplary embodiment, the binder comprises polyimide (PI). Polyimide (PI) is a polymer of imide monomers. Polyimides have been in mass production since 1955. With their high heat-resistance, polyimides enjoy diverse applications in applications demanding rugged organic materials, e.g. high temperature fuel cells, displays, and various military roles. A typical polyimide is Kapton, available from DuPont, which is produced by condensation of pyromellitic dianhydride and 4,4′-oxydianiline. Thermosetting polyimides are known for thermal stability, good chemical resistance, excellent mechanical properties, and characteristic orange/yellow color. Polyimides compounded with graphite or glass fiber reinforcements have typically flexural strengths of up to 50,000 psi (340 MPa) and flexural moduli of 3,000,000 psi (21,000 MPa). Thermoset polyimides typically exhibit very low creep and high tensile strength. These properties are maintained during continuous use to temperatures of up to 452° C. (846° F.) and for short excursions, as high as 704° C. (1,299° F.). Molded polyimide parts and laminates typically have very good heat resistance. Normal operating temperatures for such parts and laminates typically range from cryogenic to temperature exceeding 500° F. (260° C.). Polyimides are also typically inherently resistant to flame combustion and do not usually need to be mixed with flame retardants. Typical polyimide parts are not affected by commonly used solvents and oils—including hydrocarbons, esters, ethers, alcohols and freons. They also resist weak acids, but typically are not recommended for use in environments that contain alkalis or inorganic acids. Some polyimides are solvent-soluble and exhibit high optical clarity. The solubility properties of polyimides typically lend them towards spray and low temperature cure applications.

e. Polyamide-Imides (PAI)

In one exemplary embodiment, the binder comprises polyamide-imide (PAI). Polyamide-imides (PAI) may be thermosetting or thermoplastic amorphous polymers and typically have exceptional mechanical, thermal and chemical resistant properties. Exemplary polyamide-imides are produced by Solvay Specialty Polymers under the trademark Torlon. Polyamide-imides are used as wire coatings in making magnet wire. They are formed from isocyanates and TMA (trimellic acid-anhydride) in N-methyl-2-pyrrolidone (NMP). Polyamide-imides may possess desirable properties of both polyamides and polyimides, such as high strength, melt processibility, exceptional high heat capability, and broad chemical resistance. Polyamide-imide polymers can be processed into a wide variety of forms—from injection or compression molded parts and ingots—to coatings, films, fibers and adhesives. Typically, these articles reach their maximum properties with a subsequent thermal cure process.

f. Polyetherimide (PEI)

In one exemplary embodiment, the binder comprises polyetherimide (PEI). Polyetherimide (PEI) is an amorphous, amber-to-transparent thermoplastic with characteristics similar to the related plastic PEEK. Relative to PEEK, PEI is typically cheaper, but possesses lower impact strength and usable temperature. The glass transition temperature of PEI is 216° C. Its amorphous density at 25° C. is 1.27 g/cm³.

### 3. Beta-Alkoxypropionamide Solvent

The composition includes one or more solvents for entirely or partially dissolving the one or more binders discussed above. In general, due to improved dissolving power and capability of dissolved easily in water, an amide-based organic solvent can be subjected to water rinsing, and hence has desired properties as a solvent or a detergent, and can also be used as a resist peeling agent or a specific solvent for a hardly-soluble resin such as polyimide and polyamide.

The composition includes one or more β-alkoxypropionamide solvents. Beta-alkoxypropionamides, and methods of forming same, are disclosed in U.S. Pat. Nos. 8,338,645 and 8,604,240, the disclosures of which are hereby incorporated by reference in their entirety.

Beta-alkoxypropionamide solvents have the general formula (I):

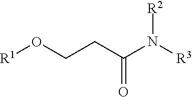

wherein R¹ is a C₁ to C₈ alkyl, and R² and R³ are independently selected from hydrogen, C₁ to C₆ alkyl, C₁ to C₆ alkoxy, C₁ to C₆ alkoxyalkyl, or glycidyl.

In one exemplary embodiment, R¹ is a C₁ to C₄ alkyl, and R² and R³ are independently selected from H or C₁ to C₄ alkyl. In a more particular embodiment, R¹ is a C₁ to C₄ alkyl, and R² and R³ are independently selected from H or CH₃. In another more particular embodiment, R¹ is a C₁ to C₄ alkyl, and R² and R³ are each CH₃.

In one exemplary embodiment, R¹ is CH₃, and R² and R³ are independently selected from H or C₁ to C₄ alkyl. In a more particular embodiment, R¹ is CH₃, and R² are R³ are independently selected from H or CH₃. In another more particular embodiment, R¹ is CH₃, and R² and R³ are each CH₃.

In one exemplary embodiment, R¹ is C₄ alkyl, and R² and R³ are independently selected from H or C₁ to C₄ alkyl. In a more particular embodiment, R¹ is C₄ alkyl, and R² are R³ are independently selected from H or CH₃. In another more particular embodiment, R¹ is C₄ alkyl, and R² and R³ are each CH₃.

In one exemplary embodiment, the solvent is 3-methoxy-N,N-dimethylpropanamide, which has the following formula (II):

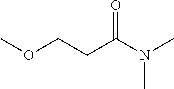

In one exemplary embodiment, the solvent is 3-buthoxy-N,N-dimethylpropanamide, which has the following formula (III):

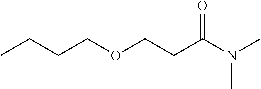

In one exemplary embodiment, the solvent is selected from the group consisting of 3-methoxy-N,N-dimethylpropanamide and 3-buthoxy-N,N-dimethylpropanamide.

In one exemplary embodiment, the composition comprises, on a wet basis, one or more β-alkoxypropionamide solvents in an amount from as little as 1 wt. %, 5 wt. %, 10 wt. %, as great as 25 wt. %, 50 wt. %, 90 wt. %, or within any range defined between any two of the foregoing values.

Beta-alkoxy propanamides, such as 3-methoxy-N,N-dimethylpropanamide, are typically colorless liquids with mild odor, that provide suitable properties for the formulation of compositions for the formation of reduced friction or non-stick coatings. 3-methoxy-N,N-dimethylpropanamide is capable of dissolving resins such as PAI or PES, is water miscible in any ratio, has a high flash point (93° C.) and provides some additional desirable features compared to other traditionally used N-alkyl-2-pyrrolidone solvents such as N-methyl-2-pyrrolidone (NMP) or N-ethyl-2-pyrrolidone (NEP). Illustrative desirable features include a lesser destabilizing effect on PTFE aqueous dispersions, lower viscosity polymer solutions, better wetting behavior against powders that allow a better and faster grinding, and a better leveling effect for some dry lubricants such as PTFE micropowders.

Some industrially available grades of solvents that correspond to the above mentioned molecules include Equamide M100, a 3-methoxy-N,N-dimethylpropanamide, and Equamide B100, a 3-buthoxy-N,N-dimethylpropanamide, each available from Idemitsu Kosan.

In some embodiments, these solvents provide a highly desirable candidate replacement for polar aprotic solvents traditionally adopted in the formulation of coatings involving engineering polymers. As shown in Table 1 below, 3-methoxy-N,N-dimethylpropanamide and N-methyl-2-pyrrolidone share similar physical properties:

TABLE 1
Comparison of NMP and 3-methoxy-N,N-dimethylpropanamide

| | 3-methoxy-N,N- | N-methyl- |
|---|---|---|
| | dimethylpropanamide | 2-pyrrolidone |
| Property | CAS: 53185-52-7 | CAS: 872-50-4 |
| Boiling point (° C.) | 216 | 204 |
| Melting point (° C.) | −49 | −24 |
| Density (20° C. g/Cm3) | 0.99 | 1.03 |
| Viscosity (20° C.; mPa · s) | 2.3 | 1.8 |
| Surface tension (23° C., nN/m) | 34.2 | 38.6 |
| Vapor pressure (20° C.; kPa) | 0.016 | 0.04 |
| Solubility parameter | 10.5 | 11.5 |
| Flash point (° C.) | 93 | 91 |
| Water compatibility | complete | complete |

### 4. Optional Components

In some embodiments, the composition may optionally include one or more additional components. Exemplary additional components include pigments, additional solvents, functional fillers, additives, and acidic or alkaline additives.

a. Pigments and Colorants

In one exemplary embodiment, the composition comprises one or more pigments, colorants, or color centers in general of any kind. In one exemplary embodiment, the composition does not include any pigments, colorants, or color centers. In one exemplary embodiment, the composition comprises, on a wet basis, one or more pigments or colorants in an amount from as little as 0 wt. %, 0.1 wt. %, 1 wt. %, 5 wt. %, 10 wt. %, as great as 25 wt. %, 50 wt. %, 75 wt. %, or within any range defined between any two of the foregoing values. In one exemplary embodiment, the coating formed from the composition comprises, on a dry basis, one or more pigments or colorants in an amount from as little as 0 wt. %, 0.1 wt. %, 1 wt. %, 5 wt. %, 10 wt. %, as great as 25 wt. %, 50 wt. %, 75 wt. %, 90 wt. %, or within any range defined between any two of the foregoing values.

b. Other Solvents

In one exemplary embodiment, the composition comprises one or more solvents in addition to the β-alkoxypropionamide solvents. In one exemplary embodiment, the composition does not include any additional solvent. Exemplary additional solvents include organic solvents and water. In one exemplary embodiment, the composition comprises, on a wet basis, one or more non-β-alkoxypropionamide solvents in an amount from as little as 0 wt. %, 5 wt. %, 10 wt. %, as great as 25 wt. %, 50 wt. %, 75 wt. %, 90 wt. %, or within any range defined between any two of the foregoing values.

c. Functional Fillers

In one exemplary embodiment, the composition comprises one or more functional fillers. Functional fillers may provide corrosion protection, higher filling factor, surface texturing, improved hardness or other features. Exemplary functional fillers include silicon carbide, barium sulphate, pyrogenic silica, wollastonite, alumina, talc, mica, silica, zinc phosphates, aluminium phosphates, waxes, and the like. In one exemplary embodiment, the composition does not include any functional fillers. In one exemplary embodiment, the composition comprises, on a wet basis, one or more functional fillers in an amount from as little as 0 wt. %, 5 wt. %, 10 wt. %, as great as 25 wt. %, 50 wt. %, 75 wt. %, or within any range defined between any two of the foregoing values. In one exemplary embodiment, the coating formed from the composition comprises, on a dry basis, one or more functional fillers in an amount from as little as 0 wt. %, 5 wt. %, 10 wt. %, as great as 25 wt. %, 50 wt. %, 75 wt. %, 90 wt. %, or within any range defined between any two of the foregoing values.

d. Formulation Additives

In one exemplary embodiment, the composition comprises one or more additives. Exemplary additives include defoamers, surface wetting agents, flow agents, pigment wetting additives, thickeners, fillers, and additives that change the electric conductivity or other features. In one exemplary embodiment, the composition does not include any formulation additives. In one exemplary embodiment, the composition comprises, on a wet basis, one or more additives in an amount from as little as 0 wt. %, 0.01 wt. %, 0.05 wt. %, as great as 1 wt. %, 5 wt. %, 10 wt. %, 30 wt. %, or within any range defined between any two of the foregoing values.

e. Acidic or Alkaline Additives

In one exemplary embodiment, the composition comprises one or more acidic or alkaline additives. In one exemplary embodiment, the composition does not include any acidic or alkaline additives. Acidic and alkaline additives may act as correctors of pH or flash rust inhibitors. Exemplary acidic and alkaline additives include dimethylethanolamine, methylamine, dimethylamine, triethanolamine, triethylamine, aminomethoxypropanol, diisopropylamine, ammonia, acetic acid, formic acid, citric acid and the like. In one exemplary embodiment, the composition comprises, on a wet basis, one or more acidic or alkaline additives in an amount from as little as 0 wt. %, 1 wt. %, 5 wt. %, as great as 10 wt. %, 15 wt. %, 20 wt. %, or within any range defined between any two of the foregoing values.

### 5. Substrates

In some exemplary embodiments, the coating composition is applied to the surface of a substrate. In one exemplary embodiment, the substrate is selected from the group consisting of metals, ceramic materials, plastics, composites, and minerals. Exemplary metals include stainless steel, aluminum, and carbon steel. Exemplary ceramic materials include glasses like borosilicate glass, porcelain enamels, various fired clays and other refractory materials. Exemplary plastics and composites include high melting point plastics and composites, such as plastics having a melting point higher than the cure temperature of the coating formulation, including polyester, polypropylene, ABS, polyethylene, carbon fiber epoxy composites, and glass fiber epoxy composites. Exemplary minerals include micas, basalts, aluminas, silicas, and wollastonites, marble and granite. In some exemplary embodiments, the substrate is a portion of a pan or other article of cookware.

The substrate may be a rigid substrate or a flexible substrate. Exemplary rigid substrates include cookware, bakeware, molds, small electrical appliances, fasteners, reprographic rollers, bearings, engine piston skirts, and other suitable substrates. Exemplary flexible substrates include glasscloth of the type commonly used in applications such as food conveyer belts for continuous ovens, architectural fabrics of the type used in stadium roofs and radar domes, as well as heat sealing belts, circuit boards, cooking sheets, and tenting fabrics, for example. “Glasscloth” or “glass cloth” is a textile material made of woven fibers such as, for example, linen, glass, or cotton. Other flexible substrates that may be coated with the present coating compositions include any material including natural or synthetic fibers or filaments, including staple fiber, fiberfill, yarn, thread, textiles, nonwoven fabric, wire cloth, ropes, belting, cordage, and webbing, for example. Exemplary fibrous materials which may be coated with the present coating compositions include natural fibers, such as vegetable, animal, and mineral fibers, including cotton, cotton denim, wool, silk, ceramic fibers, and metal fibers, as well as synthetic fibers, such as knit carbon fabrics, ultra-high molecular weight polyethylene (UHMWPE) fibers, poly(ethylene terephthlalate) (PET) fibers, para-aramid fibers, including poly-paraphenylene terephtalamide or Kevlar®, and meta-aramid fibers, such as Nomex®, each available from E.I. du Pont de Nemours and Company, polyphenylene sulfide fibers, such as Ryton®, available from Chevron Phillips Chemical Co., polypropylene fibers, polyacrylic fibers, polyacrylonitrile (PAN) fibers, such as Zoltek®, available from Zoltek Corporation, polyamide fibers (nylon), and nylon-polyester fibers, such as Dacron®, available from Invista North America.

### 6. Method of Coating

The coating composition can be prepared by any standard formulation technique such as simple addition and low shear mixing. The coating composition may be applied directly to the substrate as a base layer or primer, or may be applied over a basecoat or primer and/or a midcoat by any known technique, such as spray coating, curtain coating and roller coating, and is then cured to provide a coated substrate with a coating having improvements in gloss, non-stick performance, and abrasion and scratch resistance. Typically, basecoats will be applied by spray coating, curtain coating and roller coating, while midcoats and topcoats will be applied by roller coating. The particular compositions of the primer and/or midcoat may vary widely, and are not thought to be critical with respect to the improved properties demonstrated by the coatings disclosed herein.

In one exemplary embodiment, the coating composition is applied to the substrate, followed by drying. In an illustrative embodiment, drying may take place at a drying temperature as low as 40° C., 50° C., 60° C., 70° C., 80° C., 95° C., 100° C., as high as 105° C., 110° C., 115° C., 120° C., 125° C., or higher. In an illustrative embodiment, drying may comprise drying at the drying temperature for as little as 0.5 min, 1 min, 2 min, as long as 3 min, 5 min, 10 min, or longer. In one exemplary embodiment, the coating composition is dried by air drying at ambient temperature.

In one exemplary embodiment, the coating composition is heat cured to the substrate. In an illustrative embodiment, curing may take place at a curing temperature as low as 220° C., 250° C., 300° C. 350° C., 400° C., as high as 410° C., 420° C. 430° C., 440° C., or 450° C. In an illustrative embodiment, curing may comprise curing at the curing temperature for as little as 3 min, 5 min, 10 min, as long as 15 min, 20 min, or longer. In one exemplary embodiment, the coating composition is cured by air curing at ambient temperature.

The present coatings are typically applied to a dry film thickness (DFT) of as little as less than 5 microns, 5 microns, 10 microns, 15 microns, 20 microns, as thick as 30 microns, 40 microns, 60 microns, or thicker, depending on the application.

### 8. Exemplary Coating Formulations

Exemplary coating compositions according to the present disclosure may be an undercoat. The undercoat may be a basecoat, which is a coating applied directly to an underlying substrate (sometimes referred to as a primer). The present coating compositions may also be overcoats, which are applied over an underlying undercoat. In these embodiments, the present coating compositions may take the form of a midcoat, in which the coating is applied over an underlying undercoat and beneath a covering coating or topcoat, or the present coating compositions may take the form of a topcoat, in which the coating is applied over an underlying undercoat with the coating remaining exposed to the external environment. In other embodiments, the present coating composition may be applied directly to a substrate to form a single-layer coating in direct contact with the substrate whereby the coating is not applied over any undercoats with the coating remaining exposed to the external environment.

On a wet basis, exemplary undercoats or primers may include as little as 5 wt. %, 7 wt. %, or 10 wt. % or as great as 12 wt. %, 15 wt. % or 20 wt. % fluoropolymer, or within any range between any two of the foregoing values, such as 5-20 wt. %, 7-15 wt. %, or 10-12 wt. %, for example. Such coatings may additionally include as little as 5 wt. %, 7 wt. %, or 10 wt. % or as great as 12 wt. %, 15 wt. % or 20 wt. % binder, or within any range between any two of the foregoing values, such as 5-20 wt. %, 7-15 wt. %, or 10-12 wt. %, for example. Such coatings may further include as little as 15 wt. %, 17 wt. %, or 20 wt. % or as great as 22 wt. %, 25 wt. %, 30 wt. %, or 35 wt. % solvents, or within any range between any two of the foregoing values, such as 15-35 wt. %, 17-30 wt. %, or 20-25 wt. %, for example. The reminder of such coating compositions may include water and/or one or more fillers, pigments, or other additives.

On a wet basis, exemplary midcoats may include as little as 7 wt. %, 10 wt. %, or 12 wt. % or as great as 15 wt. %, 18 wt. % or 20 wt. % fluoropolymer, or within any range between any two of the foregoing values, such as 7-20 wt. %, 10-18 wt. %, or 12-15 wt. %, for example. Such coatings may additionally include as little as 1 wt. %, 2 wt. %, or 3 wt. % or as great as 5 wt. %, 7 wt. % or 10 wt. % binder, or within any range between any two of the foregoing values, such as 1-10 wt. %, 2-7 wt. %, or 3-5 wt. %, for example. Such coatings may further include as little as 10 wt. %, 12 wt. %, or 15 wt. % or as great as 20 wt. %, 22 wt. % or 25 wt. % solvents, or within any range between any two of the foregoing values, such as 10-25 wt. %, 12-22 wt. %, or 15-20 wt. %, for example. The reminder of such coating compositions may include water and/or one or more fillers, pigments, or other additives.

An exemplary one-coat water borne dry film lubricant composition may include as little as 3 wt. %, 5 wt. %, or 7 wt. % or as great as 9 wt. %, 11 wt. % or 15 wt. % fluoropolymer, or within any range between any two of the foregoing values, such as 3-15 wt. %, 5-13 wt. %, or 7-9 wt. %, for example. Such coatings may additionally include as little as 5 wt. %, 7 wt. %, or 10 wt. % or as great as 12 wt. %, 15 wt. % or 20 wt. % binder, or within any range between any two of the foregoing values, such as 5-20 wt. %, 7-15 wt. %, or 10-12 wt. %, for example. Such coatings may further include as little as 25 wt. %, 35 wt. %, or 45 wt. % or as great as 55 wt. %, 65 wt. %, 75 wt. % solvents, or within any range between any two of the foregoing values, such as 25-75 wt. %, 35-65 wt. %, or 45-55 wt. %, for example. The reminder of such coating compositions may include water and/or one or more fillers, pigments, or other additives.

An exemplary one-coat solvent borne dry film lubricant composition may include as little as 5 wt. %, 8 wt. %, or 10 wt. % or as great as 15 wt. %, 17 wt. % or 20 wt. % fluoropolymer, or within any range between any two of the foregoing values, such as 5-20 wt. %, 8-17 wt. %, or 10-15 wt. %, for example. Such coatings may additionally include as little as 8 wt. %, 10 wt. %, or 12 wt. % or as great as 16 wt. %, 18 wt. % or 20 wt. % binder, or within any range between any two of the foregoing values, such as 8-20 wt. %, 10-18 wt. %, or 12-16 wt. %, for example. Such coatings may further include as little as 55 wt. %, 60 wt. %, or 65 wt. % or as great as 75 wt. %, 80 wt. %, 85 wt. % solvents, or within any range between any two of the foregoing values, such as 55-85 wt. %, 60-80 wt. %, or 65-75 wt. %, for example. The reminder of such coating compositions may include water and/or one or more fillers, pigments, or other additives.

### 7. Coating Properties

Coatings prepared from the compositions described above may exhibit one or more of the following properties, together with additional properties, as evidenced by the following Examples. The coating formed from a coating composition including a β-alkoxypropionamide solvents, such as 3-methoxy-N,N-dimethylpropanamide or 3-buthoxy-N,N-dimethylpropanamide, may provide one or more improved properties compared to a coating formed from a coating composition including a N-alkyl-2-pyrrolidone solvent, such as N-methyl-2-pyrrolidone (NMP) or N-ethyl-2-pyrrolidone (NEP).

a. Viscosity and Surface Tension

In one embodiment, a coating composition including β-alkoxypropionamide solvent has a lower viscosity than a comparative coating composition including an N-alkyl-2-pyrrolidone solvent. In some embodiments, a lower viscosity allows the preparation of coating compositions with a higher weight percentage of solid at a comparable viscosity, and hence providing less volatile organic component, and higher coverage, per unit of composition.

In one embodiment, a coating composition including β-alkoxypropionamide solvent has a lower surface tension than a comparative coating composition including an N-alkyl-2-pyrrolidone solvent. In some embodiments, a lower surface tension provides superior wetting ability for a given substrate, resulting in an easier coating process

b. Grinding Particle Size Distribution

In one embodiment, a coating composition including β-alkoxypropionamide solvent provides more efficient grinding of a binder and/or a functional additive in the composition than a comparative coating composition including an N-alkyl-2-pyrrolidone solvents. In some embodiments, a more efficient grinding process results in smaller ground particles, due to the solvent wetting and de-agglomerating the binder and/or functional additive during the grinding process, which results in a reduced particle size distribution.

c. Distribution of Functional Additive on Substrate

In one embodiment, a coating formed from a composition including 3-alkoxypropionamide solvent results in a coating surface containing more functional additive than a coating formed from a comparative composition including an N-alkyl-2-pyrrolidone solvent. In some embodiments, the higher level of functional additive at the coating surface allows for the use of coatings containing less total dry lubricant compared to a comparative composition in order to achieve a similar amount of functional additive at the surface of the resultant coating.

d. Compatibility with Water-Borne Ingredients

In one embodiment, a coating formed from a composition including 3-alkoxypropionamide solvent is compatible with other water borne ingredients for the formulation of additional compositions, such as water dispersions of PTFE.

e. Release Test

In one embodiment, a coating formed from a composition including 3-alkoxypropionamide solvent results in a coating surface containing having similar or better release characteristics than a coating formed from a comparative composition including an N-alkyl-2-pyrrolidone solvent.

In one exemplary embodiment, release characteristics are determined using a milk burn test. In the milk burn test, the coated substrate is placed on a gas burner, pour 20 ml of milk are poured on the surface, and left to become dry and brown (burn). The coated substrate is placed under a stream of cold water and rated as to how the milk releases.

In one exemplary embodiment, release characteristics are determined using a dry egg release test. In the dry egg release test, the coated substrate is placed on a gas burner, and heated to 150° C. An egg is broken and placed on the surface and cooked for 2 minutes, followed by turning the egg and cooking for 1 minute. The egg is removed with a spatula, and the coated substrate is rated as to how the egg releases.

f. Corrosion Test:

In one embodiment, a coating formed from a composition including β-alkoxypropionamide solvent results in a coating surface containing having similar or better corrosion resistance than a coating formed from a comparative composition including an N-alkyl-2-pyrrolidone solvent. In one exemplary embodiment, the corrosion resistance is determined by boiling a solution of deionized water and 10 wt. % salt on the coated surface for 24 hours. The article passes the test if no defects show up on the surface.

g. Wear Testing

In one embodiment, a coating formed from a composition including (3-alkoxypropionamide solvent results in a coating surface containing having similar or better wear resistance than a coating formed from a comparative composition including an N-alkyl-2-pyrrolidone solvent.

In one exemplary embodiment, the wear resistance is determined by an LGA scrubbing test. LGA testing is divided in three phases: Wear/shaker test, scratch resistance test (with Erichsen pen) and non-stick properties after wear test. The LGA evaluation is based on the overall resistance of the coating system.

In one exemplary embodiment, the wear resistance is determined by a reciprocating abrasion test (RAT). This test measures the resistance of coatings to abrasion by a reciprocating Scotch-Brite pad. Scotch-Brite pads are made by 3M Company, Abrasive Systems Division, St. Paul, Minn. 55144-1000. Pads come in grades with varying levels of abrasiveness as follows: Lowest—7445, 7448, 6448, 7447, 6444, 7446, 7440, 5440—Highest. A Scotch-Brite 7447 pad was used and changed every 1000 cycles. The test subjects a coating to abrasion in a back and forth motion. The test is a measure of the useful life of coatings that have been subjected to scouring and other similar forms of damage caused by cleaning. TM 135C is specific to a test apparatus built by Whitford Corporation of West Chester, Pa. However, it is applicable to similar test methods such as the one described in British Standard 7069-1988.

A test machine capable of holding a 2 inch Scotch-Brite abrasive pad of a specific size to the surface to be tested with a fixed 3 kg force and capable of moving the pad in a back and forth (reciprocating) motion over a distance to 10-15 cm (4 to 6 inches). The force and motion are applied by a free falling, weighted stylus. The machine is equipped with a counter. The coated substrate is secured under the reciprocating pad by firmly fastening with bolts, clamps or tape. The part should be as flat as possible and long enough so that the pad does not run off an edge.

The abrasive pad is then cycled back and forth (one back-and-forth trip is defined as 1-cycle), and the machine was allowed to run for 1000 cycles. After 1000 cycles, the pad was replaced with a fresh pad. The test was run until 10% of the abraded area was exposed to bare metal. The abrasion resistance is reported as number of cycles per thousandth inch of coating (cycles/mil).

In one exemplary embodiment, the wear resistance is determined by a Scratch Adhesion “Happy Flower” Test (HFT). The test is performed using a pen tip affixed to a balance arm calibrated with a specific weight, the article is put on a revolving heated turntable (150° C., oil filled). After 2 hours the test stops and the score is assigned accordingly to the degree of surface damage.

In one exemplary embodiment, the wear resistance is determined by a life cycle test. The test is performed by running repeated dry abrasion (21N weight) and release or non-stick (burnt milk) cycles. The test ends when the release score reaches a value of zero.

h. Shear Stability

In one embodiment, a formulation for forming a coating including (3-alkoxypropionamide solvent has a higher shear stability than a similar formulation including an N-alkyl-2-pyrrolidone solvent.

### EXAMPLES

The following non-limiting Examples illustrate various features and characteristics of the present invention, which is not to be construed as limited thereto. Throughout the Examples and elsewhere herein, percentages are by weight unless otherwise indicated.

### Example 1

### Dissolution of PESU in 3-methoxy-N,N-dimethylpropanamide and NEP

Solutions of Veradel 3600P PESU resin from Solvay Advanced polymers were made in Equamide M100 (3-methoxy-N,N-dimethylpropanamide) and 1-ethyl-2-pyrrolidone (NEP), according to the amounts provided in Table 2:

TABLE 2
Formulation for Example 1

| | Ex. 1 | Comp. Ex. 1 |
|---|---|---|
| Veradel 3600P CAS: 25608-63-3 | 30% | 30% |
| Equamide M100 CAS: 53185-52-7 | 70% | — |
| NEP CAS: 2687-91-4 | — | 70% |

In both cases after simple stirring, a clear solution was obtained, showing that Equamide has an equally good solvency for PESU polymers as that of NEP.

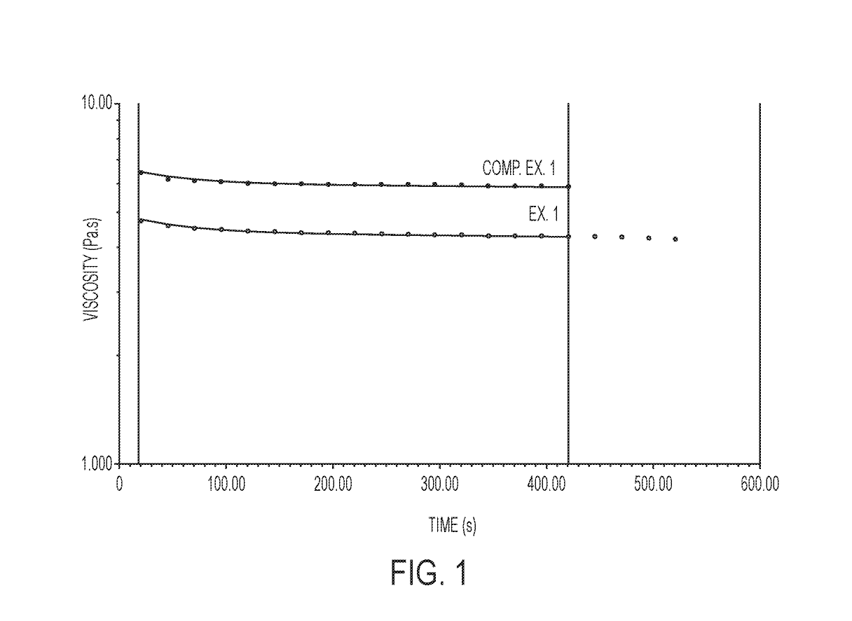

As shown in FIG. 1, and Table 3, the viscosity of each sample was determined. In both cases the viscosity has been found to have a nearly Newtonian behavior.

TABLE 3
Viscosity for Example 1

| Formula | Viscosity (Pa · s) | Surface tension (mN/m) |
|---|---|---|
| Ex. 1 | 4.319 | 34.38 |
| Comp. Ex. 1 | 5.919 | 35.56 |

The Equamide M100 sample was shown to have a lower viscosity than that of the NEP formulation, indicating that the solvency of Equamide is intrinsically superior to the solvency of NEP for this particular polymer. The superior solvency provides for the preparation of coating compositions with higher percent solids at a comparable viscosity, and hence providing less volatile organic component in percent, and higher coverage.

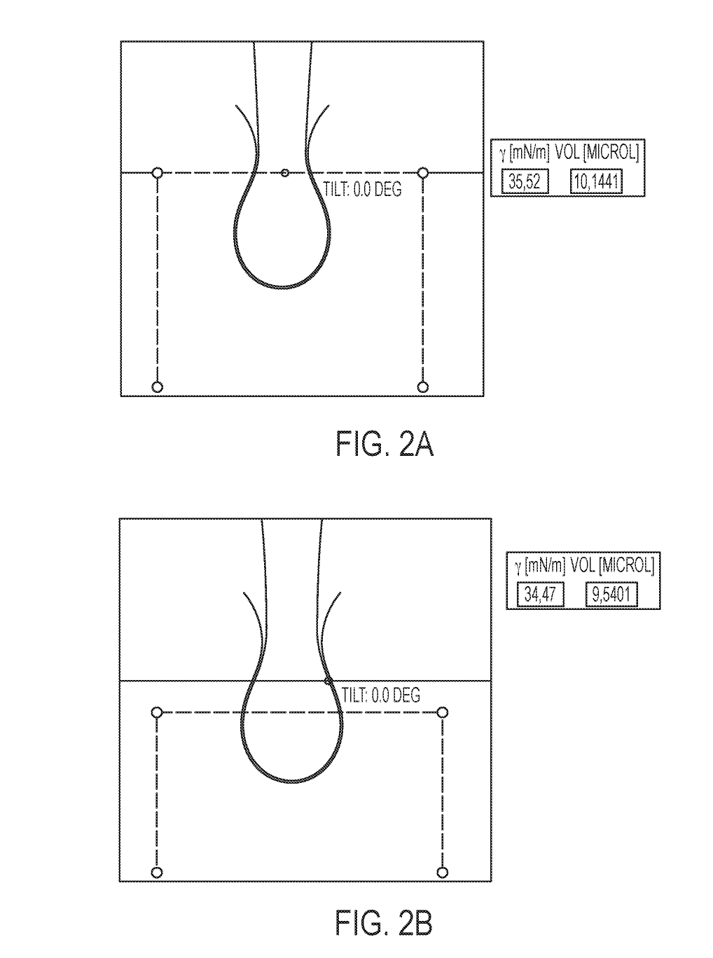

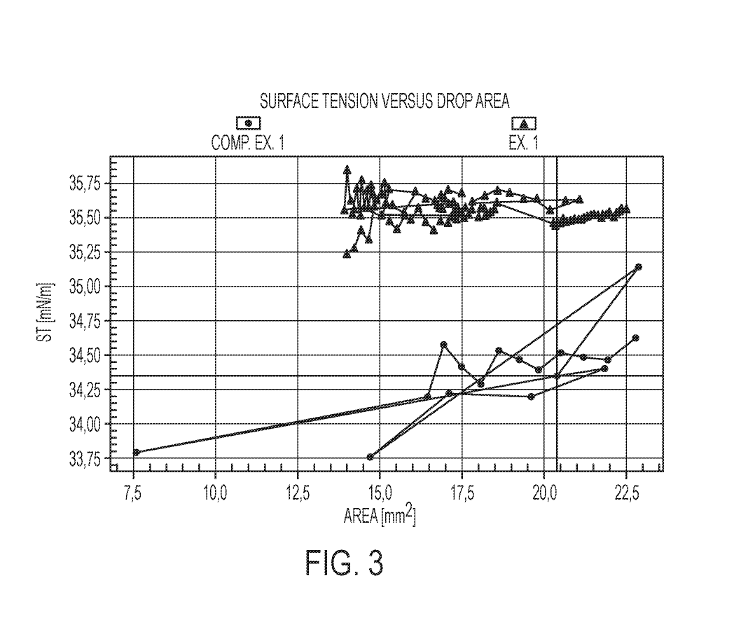

Referring next to FIGS. 2A and 2B, the surface tension of each formulation was determined. As shown in FIG. 3, the Equamide M100 formulation was shown to have a slightly lower surface tension than the NEP formulation. The lower surface tension provides for a superior wetting ability for the Equamide solution, resulting in an easier coating process.

### Example 2

### Coating Compositions Including Micropowders of PTFE

A first set of coating compositions was prepared by grinding a PTFE micropowder into formula Ex. 1 from Example 1 above. The selected PTFE micropowders were ULTRAFLON MP8-T, available from Laurel Products LLC, and Dyneon TF9207Z, available from 3M. A second set of coating compositions was prepared by grinding a PTFE micropowder into formula Comp. Ex. 1 from Example 1 above. Extra solvent was added to each formulation in order to account for the introduction of solid fillers in the shape of PTFE micropowders.

The total formulations are provided in Table 4 in grams and in Table 5 in total weight percent for each component.

TABLE 4
Formulations in grams

| | Ex. 2 | Comp. Ex. 2 | Ex. 3 | Comp. Ex. 3 |
|---|---|---|---|---|
| Ex. 1 | 800 g | — | 800 g | — |
| Comp. Ex. 1 | — | 800 g | — | 800 g |
| Equamide | 200 g | — | 200 g | — |
| M100 | | | | |
| NEP | — | 200 g | — | 200 g |
| PTFE TF9207Z | 73.6 g | 73.6 g | — | — |
| PTFE MP8-T | — | — | 73.6 g | 73.6 g |
| Total | 1073.6 g | 1073.6 g | 1073.6 g | 1073.6 g |

TABLE 5
Formulations in weight percent

| | Ex. 2 | Comp. Ex. 2 | Ex. 3 | Comp. Ex. 3 |
|---|---|---|---|---|
| Veradel 3600P | 22.36% | 22.36% | 22.36% | 22.36% |
| Equamide M100 | 70.79% | — | 70.79% | — |
| NEP | — | 70.79% | — | 70.79% |
| PTFE TF9207Z | 6.85% | 6.85% | — | — |
| PTFE MP8-T | — | — | 6.85% | 6.85% |
| Total | 100% | 100% | 100% | 100% |

The micropowders were dispersed under a Cowles impeller for few minutes, and then milled in the solution under a lab scale attritor mill operated with glass beads of about 2 mm diameter for 10 hours, under cooling jacket to prevent overheating and solvent evaporation.

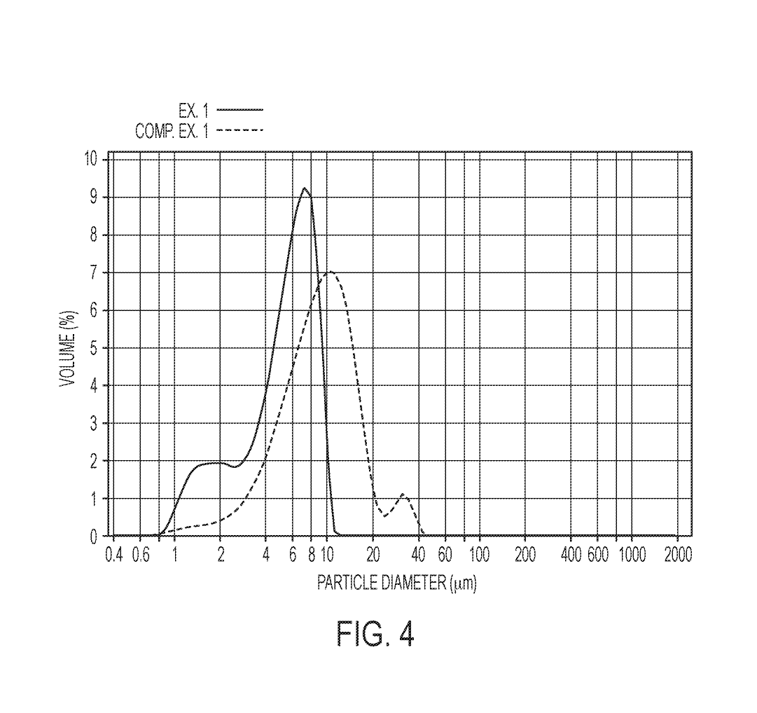

Referring to FIG. 4, the particle size distribution of Ex. 3, including the Equamide solvent, and Comp. Ex. 3, including the NEP solvent, was determined following 10 hours grinding in the attritor mill. Table 6 summarizes the particle size distribution results of FIG. 4.

TABLE 6
Particle size distribution

| | Mean | Median | S.D. | | Mode | d10 | d50 | d90 |
|---|---|---|---|---|---|---|---|---|
| | μm | μm | μm | C.V. | μm | μm | μm | μm |
| Ex. 2 | 5.41 | 5.59 | 2.46 | 45.5% | 7.08 | 1.74 | 5.59 | 8.63 |
| Comp. | 10.3 | 9.13 | 6.34 | 61.5% | 10.3 | 4.02 | 9.13 | 16.9 |
| Ex. 2 | | | | | | | | |

As shown in FIG. 4 and Table 6, the PTFE micropowder is ground much more efficiently in the Equamide solvent than in NEP. After 10 hours of grinding in the Equamide solvent, an average particle is about half of the particle size of an average particle ground in the NEP solvent. This experiment shows that a β-alkoxypropionamide solvent, such as Equamide M100, when used in combination with an engineering polymer like PESU and a PTFE micropowder, has the ability to better wet the micropowder and de-agglomerate it during the grinding process. This in turn makes the grinding process more efficient and the overall final coating composition more effective due to the reduced particle size distribution.

Formulations Ex. 3 and Comp. Ex. 3 were applied by conventional spraying on a glass panel, and the panel was first dried at 120° C. for 5 minutes, and subsequently cured at 420° C. for 5 minutes.

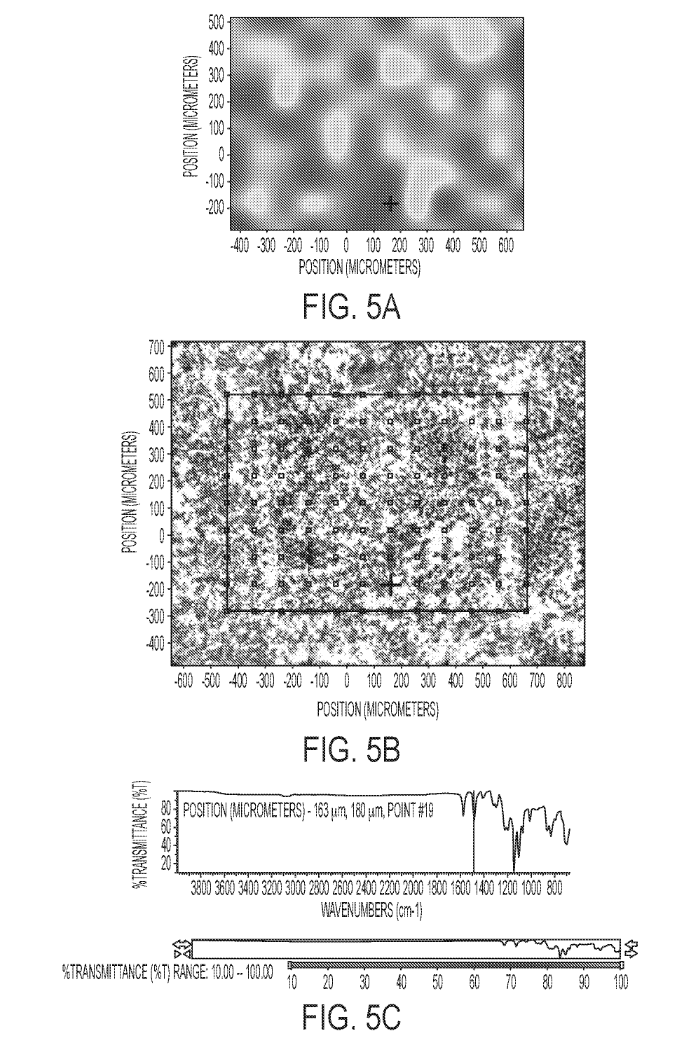

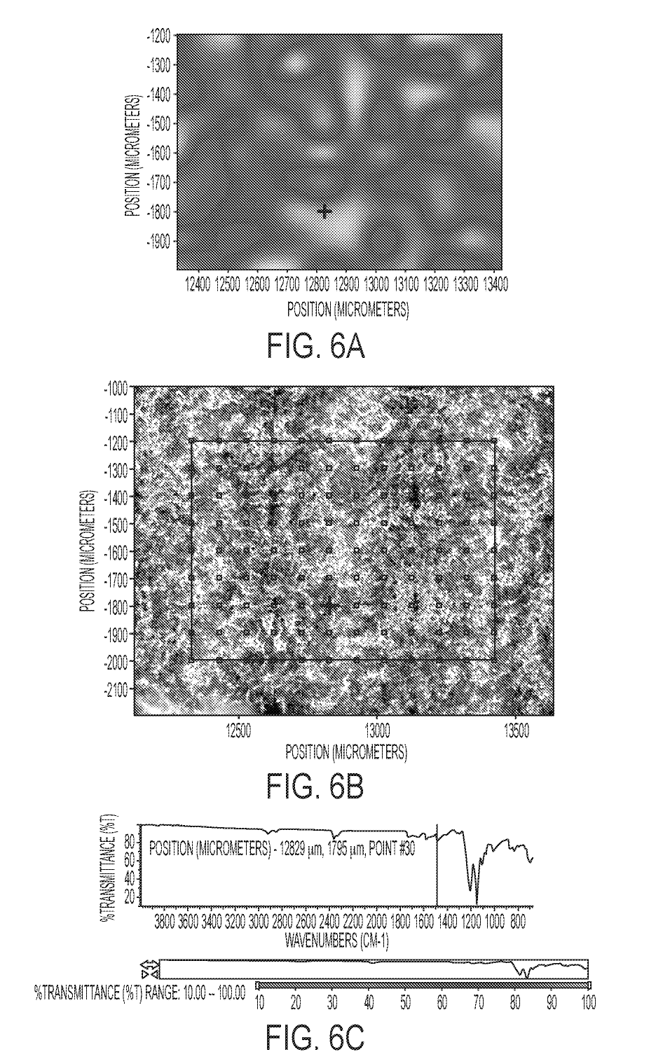

Referring to FIGS. 5 and 6, the resulting coating was analyzed under a FTIR microscope (Nicolet IN-10) with micro ATR technique for surface molecular analysis. FIGS. 5A-5C show the spectral maps for the NEP formulation Comp. Ex. 3, and FIGS. 6A-6C show the spectral map for the Equamide formulation Ex. 3. The collected spectral maps account for the surface saturation of PTFE vs. PESU. By normalizing the collected spectra for the strongest peak at 1148 cm⁻¹ typical of PTFE, the relative strength of the 1481 cm⁻¹ peak, representative of PESU, is proportional to the surface saturation of this polymer.

The maps of the Equamide formulation Ex. 3 in FIGS. 6A-6C have a much weaker PESU surface compared to the NEP formulation Comp. Ex. 3 in FIG. 5. This indicates that the presence of Equamide solvent in place of NEP facilitates the PTFE floating on the surface of the coating, and a more homogeneous de-agglomeration of the particles, as it appears from the optical microscopy measurements taken with the same instrument. In fact, for the NEP formulation Comp. Ex. 3 in FIGS. 5A-5C the saturation of the 1481 cm⁻¹ peak reaches on average 50-60% of transmittance indicating a strong surface presence of PESU, while for the Equamide formulation Ex. 3 in FIGS. 6A-6C the same peak reaches on average only 80-90% of transmittance. This indicates for a poor surface saturation of PESU, indicating a high level of PTFE on the surface of the Ex. 3 formulation.

The higher level of PTFE on the surface for the Equamide formulation Ex. 3 suggests a more effective floating or partitioning effect induced by Equamide solvent with respect to PTFE, allowing the preparation of more effective coatings containing less dry lubricant compared to traditional composition, because less dry lubricant powder it is needed in the wet composition in order to saturate the surface of the resultant coating and provide the same level of PTFE at the surface.

### Example 3

### Shear Stability

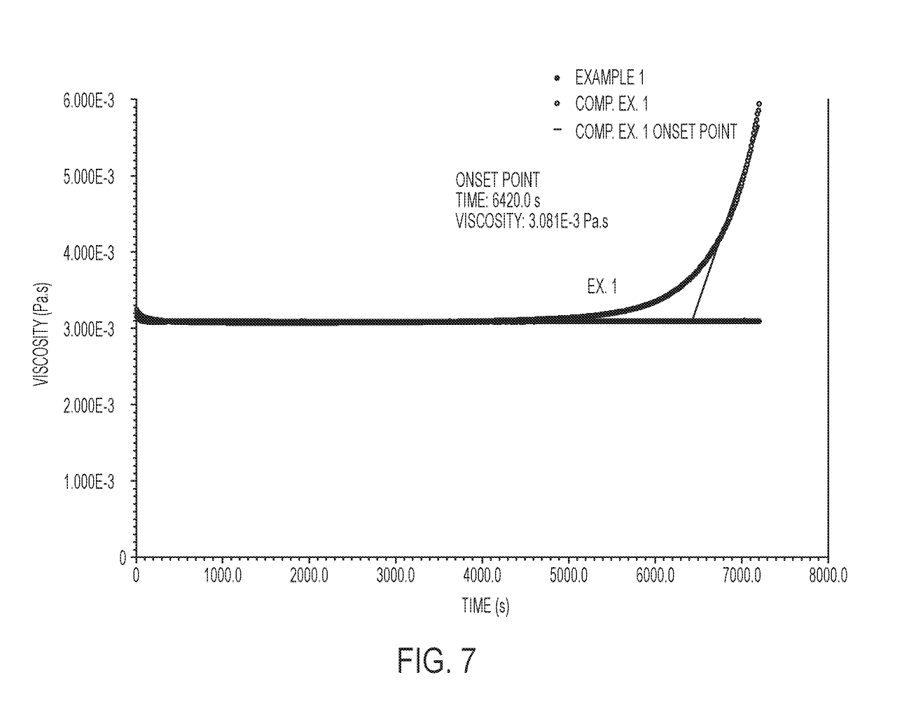

A blend of a PTFE dispersion and N-ethyl-2-pyrrolidone (NEP) was prepared. A similar blend of the PTFE dispersion and Equamide M100 (3-methoxy-N,N-dimethylpropanamide) was also prepared. Both blends were subject to a shear stability test. The shear stability test shows the destabilization imparted by the resin and its solvents to the PTFE colloidal dispersion. The results are illustrated in FIG. 7.

As shown in FIG. 7, the NEP-PTFE blend undergoes a viscosity increase after 6400 seconds, indicating gelling, while the Equamide-PTFE blend shows no viscosity drift.

Next a blend of a PTFE dispersion and a hydrolysed PAI solution prepared by Polyresin S.rl. (RO 460 INT M 364) based on Resistherm AI-244 from Bayer and N-methyl-2-pyrrolidone (NMP) was prepared with a weight ratio of 50:50. A similar blend of the PTFE dispersion and composition accordingly to EX. 4 containing Equamide M100 (3-methoxy-N,N-dimethylpropanamide) was also prepared with a weight ratio of 50:50. Two separate sets of blends were prepared with different grades of PTFE dispersions from two different suppliers: Dupont DISP 40 and GFL Inoflon AD9200.

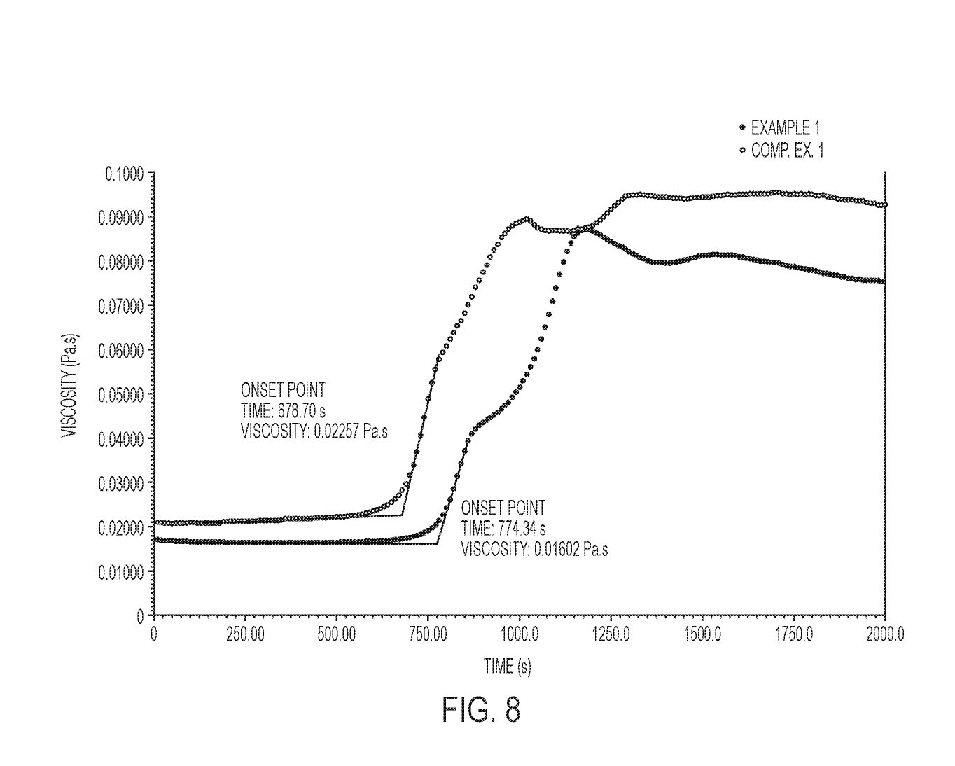

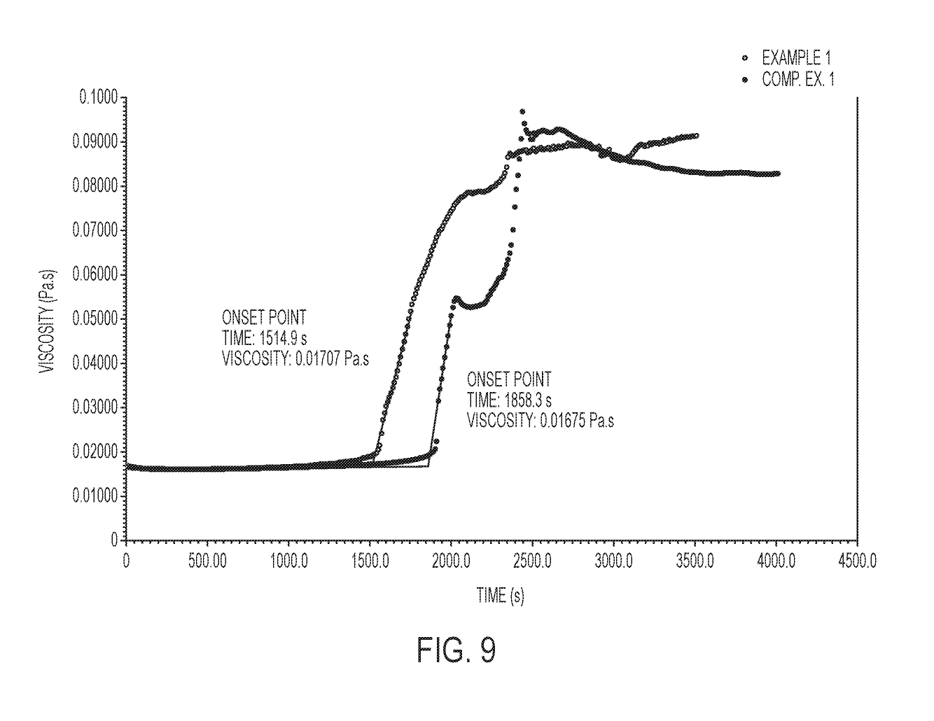

Both blends were subject to a shear stability test performed with a Rheometer TA Instruments AR2000 EX equipped with double gap cylinders geometry and operated at 50° C. at constant shear rate of 1000 s-1. The shear stability test shows the destabilization imparted by the resin and its solvents to the PTFE colloidal dispersion. FIG. 8 illustrates the results for the PTFE resin GFL Inoflon AD9200. FIG. 9 illustrates the results for the PTFE resin Dupont DISP 40.

The results of onset of gel point shown in FIGS. 8 and 9 are summarized in Table 7 below:

TABLE 7
Gel Point Results

| | GFL Inoflon | Dupont |
|---|---|---|
| | AD9200 | DISP 40 |
| Sample with Equamide M100 (3-methoxy- | 774.34 | 1858.3 |
| N,N-dimethylpropanamide) solvent | | |
| Sample with N-methyl-2-pyrrolidone (NMP) | 678.70 | 1514.9 |
| solvent | | |

As shown in FIGS. 8 and 9 and in the table, the Equamide M100-PTFE blend undergoes a viscosity increase after 774.34 seconds, and 1858 seconds respectively with Inoflon AD9200 and Dupont DISP 40 PTFE dispersions, indicating an increase of shear stability of +14% and +22% respectively compared to the NMP containing homologues.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

We claim:

1. A composition for forming a coating, the composition comprising:
at least one functional additive;
at least one binder; and
at least one β-alkoxypropionamide solvent of the formula: 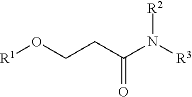
wherein R¹ is a C₁ to C₈ alkyl, and
R² and R³ are independently selected from hydrogen, C₁ to C₆ alkyl, C₁ to C₆ alkoxy, C₁ to C₆ alkoxyalkyl, or glycidyl.

2. The composition of claim 1, wherein R¹ is a C₁ to C₄ alkyl, and R² and R³ are independently selected from H or C₁ to C₄ alkyl.

3. The composition of claim 1, wherein the β-alkoxypropionamide solvent is 3-methoxy-N,N-dimethylpropanamide.

4. The composition of claim 1, wherein the β-alkoxypropionamide solvent is 3-buthoxy-N,N-dimethylpropanamide.

5. The composition of claim 1, wherein the at least one functional additive comprises at least one additive selected from the group consisting of: graphite, molybdenum disulphide, hexagonal boron nitride, fluoropolymers, and silicone-based materials.

6. The composition of claim 5, wherein the at least one functional additive includes at least one fluoropolymer selected from the group consisting of: polytetrafluorethylene (PTFE); fluorinated ethylene-propylene (FEP); perfluoroalkoxy polymer (PFA); perfluoro methylalkoxy polymer (MFA); polyvinylidene fluoride (PVDF); polyethylenetetrafluoroethlene (ETFE); polyethylenechlorotrifluoroethylene (ECTFE); and polymers of tetrafluoroethylene, hexafluoropropylene and vinylidene fluoride (THV).

7. The composition of claim 5, wherein the at least one functional additive includes polytetrafluorethylene (PTFE).

8. The composition of claim 1, wherein the at least one binder comprises at least one engineering polymer.

9. The composition of claim 8, wherein the engineering polymer comprises at least one polymer selected from the group consisting of: polyphenylene sulfide (PPS); polyetheretherketone (PEEK); polysulfone (PESU); polyimide (PI); polyamide-imides (PAI); and polyetherimide (PEI).

10. The composition of claim 1, further comprising at least one additional component selected from the group consisting of pigments and colorants; non-β-alkoxypropionamide solvents; functional fillers; defoamers; surface wetting agents; flow agents; pigment wetting additives; thickeners; fillers; additives that change the electric conductivity of the composition; pH correctors; and flash rust inhibitors.

11. A method of forming a coating comprising:
providing a composition, wherein the composition comprises
at least one functional additive;
at least one binder; and
at least one β-alkoxypropionamide solvent of the formula: 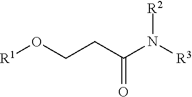
wherein R¹ is a C₁ to C₈ alkyl, and
R² and R³ are independently selected from hydrogen, C₁ to C₆ alkyl, C₁ to C₆ alkoxy, C₁ to C₆ alkoxyalkyl, or glycidyl;
applying the composition to a substrate; and
curing the composition to produce a coating.

12. The method of claim 11, wherein said curing step comprises heating the coating at a temperature of 400° C. to 450° C. for a time from 3 minutes to 20 minutes.

13. The method of claim 11, wherein said curing step comprises drying in air at ambient temperature.

14. The method of claim 11, further comprising the additional steps of:
heating the applied composition and substrate to form a first dried layer; and
applying a second composition to the first dried layer, wherein the second composition comprises at least one solvent, at least one binder, and at least one functional additive;
wherein said curing step comprises curing the first dried layer and the second composition to produce a coating.

15. The method of claim 11, wherein the β-alkoxypropionamide solvent is selected from the group consisting of 3-methoxy-N,N-dimethylpropanamide and 3-buthoxy-N,N-dimethylpropanamide.

16. The method of claim 11, wherein the at least one functional additive is selected from the group consisting of: graphite, molybdenum disulphide, hexagonal boron nitride, fluoropolymers, and silicone-based materials.

17. The method of claim 11, wherein the binder comprises at least one engineering polymer selected from the group consisting of: polyphenylene sulfide (PPS); polyetheretherketone (PEEK); polysulfone (PESU); polyimide (PI); polyamide-imides (PAI); and polyetherimide (PEI).

18. A coated article comprising a substrate coated with a coating, wherein the coating is formed from composition comprising:
at least one functional additive;
at least one binder; and
at least one β-alkoxypropionamide solvent of the formula: 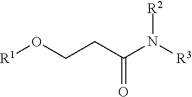
wherein R¹ is a C₁ to C₈ alkyl, and
R² and R³ are independently selected from hydrogen, C₁ to C₆ alkyl, C₁ to C₆ alkoxy, C₁ to C₆ alkoxyalkyl, or glycidyl.

19. The coated article of claim 18, wherein the substrate is selected from the group consisting of cookware, bakeware, molds, small electrical appliances, fasteners, reprographic rollers, glasscloth, architectural fabrics, as well as heat sealing belts, circuit boards, cooking sheets, tenting fabrics, staple fiber, fiberfill, yarn, thread, textiles, nonwoven fabric, wire cloth, ropes, belting, cordage, and webbing.

20. The coated article of claim 18, wherein the article is an article of cookware.

21. The coated article of claim 18, wherein the coating has a dry film thickness from 5 microns to 30 microns.
